SEQUENCE LISTING (12 sequences)

SEQ ID NO: 1 - DNA, 90 bp, synthetic construct
  source 1..90: mol_type = other DNA
gcctgttgtgagcctcctaacaaactactaactaggtaagatcacgcagcactaaacgacgtagttgccacatgcttattcttgtctccc

SEQ ID NO: 2 - DNA, 80 bp, synthetic construct
  source 1..80: mol_type = other DNA
tcgcacattccgcttctaccgggggggtcgagctgagtggatgcgaatctgtgggtgggccgtaagtccgtgtgtgcgaa

SEQ ID NO: 3 - DNA, 22 bp, synthetic construct
  source 1..22: mol_type = other DNA
tgtgagcctcctaacaaactac

SEQ ID NO: 4 - DNA, 22 bp, synthetic construct
  source 1..22: mol_type = other DNA
caagaataagcatgtggcaact

SEQ ID NO: 5 - DNA, 27 bp, synthetic construct
  source 1..27: mol_type = other DNA
aggtaagatcacgcagcactaaacgac

SEQ ID NO: 6 - DNA, 19 bp, synthetic construct
  source 1..19: mol_type = other DNA
tcgcacattccgcttctac

SEQ ID NO: 7 - DNA, 20 bp, synthetic construct
  source 1..20: mol_type = other DNA
gattcgcacacacggactta

SEQ ID NO: 8 - DNA, 25 bp, synthetic construct
  source 1..25: mol_type = other DNA
cagattcgcatccactcagctcgac

SEQ ID NO: 9 - DNA, 58 bp, synthetic construct
  source 1..58: mol_type = other DNA
cgacgatcggaccgcagtacccacccaccagccccaacatcatgcccatccgtgtgtg

SEQ ID NO: 10 - DNA, 20 bp, synthetic construct
  source 1..20: mol_type = other DNA
ataggagtcacgacgaccag

SEQ ID NO: 11 - DNA, 20 bp, synthetic construct
  source 1..20: mol_type = other DNA
tcaagaggtagacgcacata

SEQ ID NO: 12 - DNA, 80 bp, synthetic construct
  source 1..80: mol_type = other DNA
ataggagtcacgacgaccagagactaaaactcgcccagaccgtcatattgtaagttcagttatgtgcgtctacctcttga

(19) United States
(12) Patent Application Publication
(10) Pub. No.: US 2026/0152784 A1
(43) Pub. Date: Jun. 4, 2026

(54) METHOD FOR SIMULTANEOUS DETECTION OF MULTIPLE ALLERGENS IN FOOD

(71) Applicant: Zhejiang Gongshang University, Hangzhou (CN)

(72) Inventors: Linglin FU, Hangzhou (CN); Chong WANG, Hangzhou (CN); Jinru ZHOU, Hangzhou (CN); Shandong WU, Hangzhou (CN); Jian HE, Hangzhou (CN); Sufang DUAN, Hangzhou (CN); Siying ZHU, Hangzhou (CN); Juan ZHONG, Hangzhou (CN); Yi SUN, Hangzhou (CN); Zhoujie WU, Hangzhou (CN); Yi LIU, Hangzhou (CN)

(73) Assignee: Zhejiang Gongshang University, Hangzhou (CN)

(21) Appl. No.: 19/403,279

(22) Filed: Nov. 28, 2025

(30) Foreign Application Priority Data
Dec. 3, 2024 (CN) 202411763487.9

(51) Int. Cl. C12Q 1/6825; C12Q 1/6804; C12Q 1/6806; C12Q 1/686; G01N 21/64; G01N 33/02

(52) U.S. Cl. CPC C12Q 1/6825; C12Q 1/6804; C12Q 1/6806; C12Q 1/686; G01N 21/6428; G01N 33/02; C12Q 2600/16; G01N 2021/6432; G01N 2333/415

(57) ABSTRACT

A method for simultaneous detection of multiple allergens in food includes: diluting a plurality of target allergen proteins in a concentration gradient, adding them to an ELISA plate, and incubating overnight; washing the ELISA plate with a PBST washing solution to remove unbound proteins; adding 5% BSA blocking reagent to the ELISA plate, incubating with shaking, and washing away the blocking reagent; premixing aptamers of the plurality of target allergen proteins into a premixed solution; adding the premixed solution to the ELISA plate from which the blocking reagent is washed away, incubating with shaking, and washing away the premixed solution; adding ultrapure water to the ELISA plate, incubating at 95° C. to denature the allergen proteins, and recovering a supernatant containing the aptamers; using the supernatant as a DNA template, performing Taqman qPCR amplification and fluorescence signal detection to obtain a reaction ct value; and plotting a standard curve.

### CROSS REFERENCE TO THE RELATED APPLICATIONS

This application is based upon and claims priority to Chinese Patent Application No. 202411763487.9, filed on Dec. 3, 2024, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBCZL-CG006-PKG SequenceListing.xml, created on 11/26/2025, and is 11,732 bytes in size.

### TECHNICAL FIELD

This invention relates to the technical field of food allergen detection, specifically to a method for simultaneous detection of multiple allergens in food.

### BACKGROUND

The prevalence of food allergies is approximately 2% in adults and as high as 8% in children. Because even low concentrations of allergens can trigger severe immune responses, the main treatment for food allergies is currently avoiding contact with the allergens. Therefore, establishing more sensitive and accurate food allergen detection technologies is crucial. In 2013, the World Health Organization (WHO) listed milk, eggs, soybeans, peanuts, wheat, nuts, fish, and alpha-class crustaceans as the “eight major allergens in the world,” among which gliadin and Ara h1 protein are the main proteins that trigger allergic immune responses.

Currently, there are many methods for detecting allergens in food, primarily immunological methods such as enzyme-linked immunosorbent assay (ELISA) and molecular biological methods such as real-time quantitative polymerase chain reaction (PCR). These methods each have their advantages and disadvantages. While ELISA has a short detection time, the stability of the biological antibodies during the detection process is poor. Although real-time quantitative PCR can effectively overcome the influence of changes in the structure of allergen proteins, it can only target the gene sequence encoding the target protein, and the instrument operation is relatively complex and expensive. Therefore, it is necessary to develop a new method for detecting allergens in food.

### SUMMARY

The present invention aims to at least partially solve one of the technical problems in related technologies. Therefore, the purpose of the present invention is to provide a method for simultaneous detection of multiple allergens in food.

Therefore, the present invention proposes a method for simultaneous detection of multiple allergens in food, including the following steps:
(1) diluting a plurality of target allergen proteins in a concentration gradient, adding them to an ELISA plate, and incubating overnight;
(2) washing the ELISA plate with a phosphate-buffered saline containing 0.1% Tween-20 (PBST) washing solution to remove unbound proteins;
(3) adding 5% bovine serum albumin (BSA) blocking reagent to the ELISA plate, incubating with shaking, and then washing away the blocking reagent;
(4) premixing aptamers of the plurality of target allergen proteins into a premixed solution;
(5) adding the premixed solution to the ELISA plate from which the BSA blocking reagent is washed away in the step (3), incubating with shaking, and then washing away the premixed solution;
(6) adding ultrapure water to the ELISA plate, incubating at 95° C. to denature the allergen proteins, and recovering a supernatant containing the aptamers;
(7) using the supernatant containing the aptamers as a DNA template, performing Taqman quantitative polymerase chain reactions (qPCR) amplification and fluorescence signal detection to obtain a reaction ct value; and
(8) plotting a standard curve with the reaction ct value as an ordinate and a target allergen protein concentration as an abscissa.

According to the method for the simultaneous detection of the multiple allergens in the food of the present invention, this method utilizes aptamers to specifically recognize target allergen proteins. When the allergen protein denatures and alters its tertiary structure, the aptamer cannot bind to it. By measuring the aptamer content in the system, the content of the target allergen protein is indirectly reflected. Thus, by replacing the target genomic DNA in traditional qPCR methods for detecting allergen proteins with aptamer molecules, the complex and time-consuming target DNA extraction process is eliminated. The method has good sensitivity, enabling simultaneous detection of the content of multiple allergen proteins in food with high efficiency and strong specificity, and can play an important role in food allergen detection and related fields.

Optionally, the target allergen proteins include gliadin and peanut Ara h1 protein; the nucleotide sequence of an aptamer of the gliadin is shown in SEQ ID NO: 1; and the nucleotide sequence of an aptamer of the peanut Ara h1 protein is shown in SEQ ID NO: 2.

Furthermore, in the Taqman qPCR detection, a 5′ end of a Taqman probe used for the gliadin is modified with 6-carboxyfluorescein (FAM), and a 3′ end of the Taqman probe used for the gliadin is labeled with a black hole quencher 1 (BHQ1) group; a 5′ end of a Taqman probe used for the peanut Ara h1 protein is modified with VIC, and a 3′ end of the Taqman probe used for the peanut Ara h1 protein is labeled with the BHQ1 group.

Furthermore, in the Taqman qPCR detection, the sequences of primers and a probe used for the gliadin are shown in SEQ ID NOS: 3-5; and the sequences of primers and a probe used for the peanut Ara h1 protein are shown in SEQ ID NOS: 6-8.

Optionally, the plotted standard curve satisfies a condition of a correlation coefficient R²≥0.98.

Optionally, a reaction system of the Taqman qPCR amplification is as follows: total reaction volume of 20 μL, Tagman Probe Master Mix of 10 μL, 10 μM upstream primer of 0.4 μL, M downstream primer of 0.4 μL, 10 μM Tagman Probe of 0.2 μL, the DNA template of 1 μL, and sterile water of 8 μL; and
a reaction program of the Taqman qPCR amplification is as follows: denaturation at 95° C. for 1 min; the denaturation at 95° C. for 10 s, annealing and extension at 60° C. for 30 s, while simultaneously collecting fluorescence signals, for a total of 40 cycles.

Optionally, in the step (3), the blocking reagent is added and incubated with shaking for 2 h.

Optionally, in the step (5), the premixed solution is added and incubated with shaking for 1 h.

Optionally, in the step (6), the incubating at 95° C. is performed for 12 min.

The present invention also proposes an application of the method for the simultaneous detection of the multiple allergens in the food in detecting allergens in the food, after diluting a concentration of a protein to be tested, the reaction ct value is obtained using the method.

Additional aspects and advantages of the present invention will be set forth in part in the description which follows, and in part will be obvious from the description, or may be learned by practice of the present invention.

### BRIEF DESCRIPTION OF THE DRAWINGS

FIG. 1 shows a standard curve of the gliadin according to an embodiment of the present invention;

FIG. 2 shows a standard curve of peanut Ara h1 protein according to an embodiment of the present invention;

FIG. 3 shows specificity results of the gliadin according to an embodiment of the present invention;

FIG. 4 shows specificity results of peanut Ara h1 protein according to an embodiment of the present invention;

FIG. 5 shows detection results of β-lactoglobulin according to an embodiment of the present invention.

### DETAILED DESCRIPTION OF THE EMBODIMENTS

The technical solutions of the present invention are illustrated below through specific examples. It should be understood that one or more method steps mentioned in the present invention do not preclude the existence of other method steps before or after the combined steps, or the insertion of other method steps between these explicitly mentioned steps; it should also be understood that these embodiments are only for illustrating the present invention and not for limiting the scope of the present invention. Moreover, unless otherwise stated, the numbering of each method step is merely a convenient tool for identifying each method step, and not for limiting the order of each method step or the scope of implementation of the present invention. Changes or adjustments to their relative relationships, without substantially altering the technical content, should also be considered within the scope of implementation of the present invention.

To better understand the above technical solutions, exemplary embodiments of the present invention are described in more detail below. Although exemplary embodiments of the present invention are shown, it should be understood that the present invention can be implemented in various forms and should not be limited to the embodiments set forth herein. Rather, these embodiments are provided to enable a more thorough understanding of the present invention and to fully convey the scope of the present invention to those skilled in the art.

The test materials used in the present invention are all common commercially available products; unless otherwise specified, the experiments involved are conventional experimental methods.

The present invention will be described below with reference to specific embodiments. It should be noted that these embodiments are merely descriptive and do not limit the present invention in any way.

### Example 1

(1) Two target allergen proteins (gliadin and peanut Ara h1 protein) were diluted in a concentration gradient (0.1 μg/mL, 0.5 μg/mL, 1 μg/mL, 2 μg/mL, 4 μg/mL, 8 μg/mL, 12 μg/mL, 16 μg/mL, 20 μg/mL) and added to an ELISA plate, 100 μL per well, and incubated overnight.
(2) 200 μL of PBST (phosphate-buffered saline (PBS) containing 0.1% Tween-20) was added to each well of the ELISA plate, then patted dry. This process was repeated three times to remove unbound target proteins.
(3) 200 μL of 5% bovine serum albumin (BSA) blocking reagent was added to each well of the ELISA plate, and after incubation with shaking for 2 h, the plate was washed three times with PBST washing solution and patted dry.
(4) The aptamers of the two target allergen proteins were premixed to form a premixed solution with a concentration of 20 nM. The sequences of the aptamers for the gliadin and the peanut Ara h1 protein are shown in Table 1 below.

TABLE 1
Aptamer sequences of the gliadin and
the peanut Ara h1 protein

| Allergen | Name | Sequence (5′-3′) |
|---|---|---|
| Gliadin | G33 | GCCTGTTGTGAGCCTCCTAACAAAC |
| | | TACTAACTAGGTAAGATCACGCAGC |
| | | ACTAAACGACGTAGTTGCCACATGC |
| | | TTATTCTTGTCTCCC (SEQ |
| | | ID NO: 1) |
| Peanut Ara h1 | A 1 | TCGCACATTCCGCTTCTACCGGGGG |
| protein | | GGTCGAGCTGAGTGGATGCGAATCT |
| | | GTGGGTGGGCCGTAAGTCCGTGTGT |
| | | GCGAA (SEQ ID NO: 2) |

(5) The premixed solution was added to the ELISA plate after washing away the blocking reagent in step (3). 100 μL of the premixed solution was added to each well, after incubation with shaking for 1 h, the premixed solution was washed away, and the plate was washed 5 times with 200 μL PBST buffer.
(6) The ultrapure water was added to the ELISA plate, and 50 μL of MilliQ water was added to each well; the ELISA plate was incubated at 95° C. for 12 min to denature the target allergen proteins. After the plate cooled to room temperature, 30 μL of supernatant was transferred from each well of the ELISA plate to a centrifuge tube for later use.
(7) The supernatant containing the aptamer was used as a DNA template, and the Taqman qPCR amplification and the fluorescence signal detection are performed. The Taqman probe used for gliadin is modified with FAM at the 5′ end and labeled with the BHQ1 group at the 3′ end; and the Taqman probe used for peanut Ara h1 protein is modified with VIC at the 5′ end and labeled with the BHQ1 group at the 3′ end. In Tagman qPCR detection, the sequences of the probes and primers used for the gliadin and the peanut Ara h1 protein are shown in Table 2.

TABLE 2
Sequences of primers and probes

| | | | Amplified |
|---|---|---|---|
| | Primer/ | | Fragment |
| | Probe | | Size |
| Name | Name | Sequence (5′-3′) | (bp) |
| G33 | G33-F | TGTGAGCCTCCTAACA | 78 |
| | | AACTAC | |
| | | (SEQ ID NO: 3) | |
| | G33-R | CAAGAATAAGCATGT | |
| | | GGCAACT | |
| | | (SEQ ID NO: 4) | |
| | G33-P | FAM-AGGTAAGATCA | |
| | | CGCAGCACTAAACGA | |
| | | C-BHQ1 | |
| | | (SEQ ID NO: 5) | |
| A1 | A1-F | TCGCACATTCCGCTT | 82 |
| | | CTAC | |
| | | (SEQ ID NO: 6) | |
| | A1-R | GATTCGCACACACGG | |
| | | ACTTA | |
| | | (SEQ ID NO: 7) | |
| | A1-P | VIC-CAGATTCGCAT | |
| | | CCACTCAGCTCGA | |
| | | C-BHQ1 | |
| | | (SEQ ID NO: 8) | |

The reaction system of the Taqman qPCR amplification was as follows: total reaction volume of 20 μL, Tagman Probe Master Mix of 10 μL, 10 μM upstream primer of 0.4 μL, 10 M downstream primer of 0.4 μL, 10 μM Tagman Probe of 0.2 μL, DNA template of 1 μL, and sterile water of 8 μL.

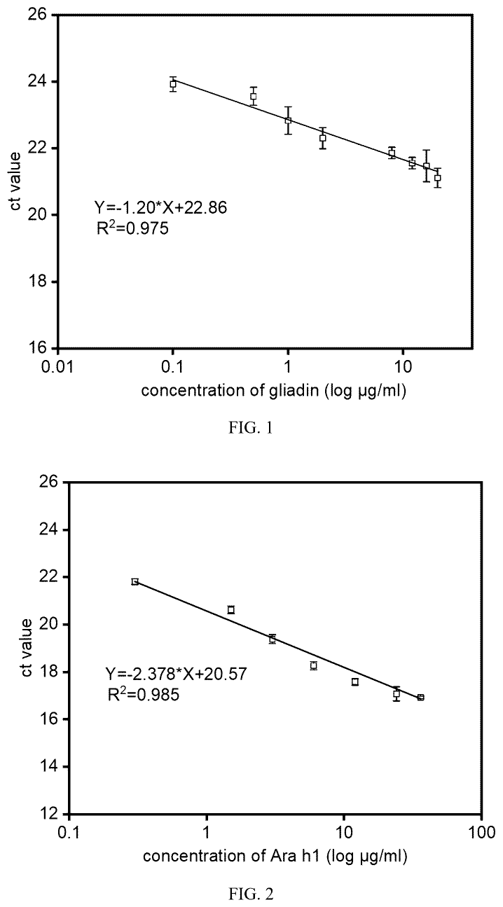

The reaction program of the Taqman qPCR amplification was: denaturation at 95° C. for 1 min; denaturation at 95° C. for 10 s, annealing and extension at 60° C. for 30 s, while simultaneously collecting fluorescence signals, for a total of 40 cycles.
(7) The reaction ct value was read, and a standard curve was plotted with ct value (reaction ct value) as the ordinate and the target allergen protein concentration (g/mL) as the abscissa. The abscissa was set to logarithmic form, and a four-parameter fitting was performed using the logistic model in Origin software. The standard curve was then plotted, as shown in FIGS. 1-2. The results show a good linear relationship between protein concentration and ct value. The correlation coefficient R² of the gliadin group is 0.975, and the linear regression equation of the standard curve is Y=−1.20*X+22.86. The correlation coefficient R² for the peanut Ara h1 group was 0.985, and the linear regression equation of the standard curve is Y=−2.38*X+20.57.

### Example 2: Specificity Test

Ovalbumin (OVA), β-lactoglobulin (β-Lg), α-casein, and a mixed group of the gliadin and the peanut Ara h1 protein (with a ratio of 1:1 for the two proteins) were diluted to 10 μg/mL. The ct value was detected using the established detection method based on aptamers and Taqman probes (Example 1). Independent samples t-test analysis was performed on the obtained data using GraphPad Prism 9 software. Significant differences are indicated by *, *p<0.05, **p<0.01, ***p<0.001, ****p<0.0001.

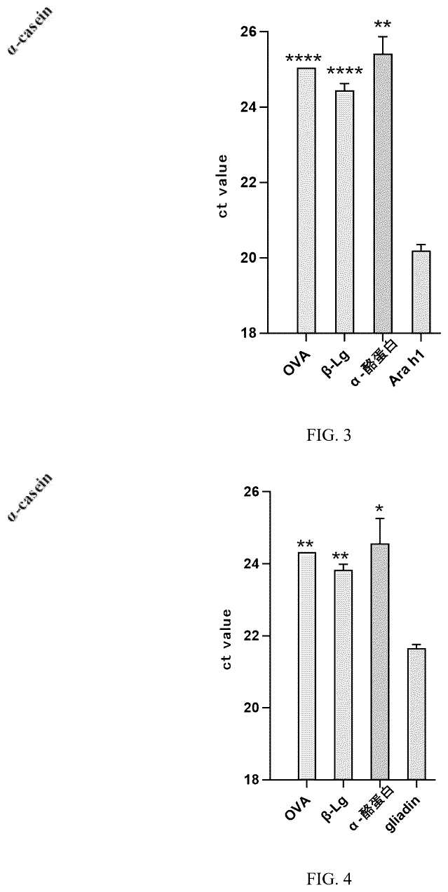

The results are shown in FIGS. 3-4, demonstrating that this method has good specificity for the gliadin and the peanut Ara h1 protein.

### Example 3 Precision Determination

The precision of the present application is affected by various factors, such as the number of plate washes and the choice of reagents. Precision is determined by evaluating intra-batch difference and inter-batch difference.
(1) Intra-batch: gliadin and peanut Ara h1 protein were mixed in a 1:1 ratio and diluted to three gradient concentrations of 5 μg/mL, 10 μg/mL, and 20 μg/mL. Five replicates were performed for each concentration. The established detection method based on aptamers and Taqman probes (Example 1) was used to detect the ct value, and the coefficient of variation (CV) was calculated.
(2) Inter-batch: gliadin and peanut Ara h1 protein were mixed in a 1:1 ratio and diluted to three gradient concentrations of 5 μg/mL, 10 μg/mL, and 20 μg/mL. The established detection method based on aptamers and Taqman probes (Example 1) was used to detect the ct value. Measurements were performed once daily for 5 consecutive days.

Intra-batch and inter-batch differences can be represented by the coefficient of variation (CV), CV=SD/X*100%, where SD is the standard deviation and X is the mean. Typically, a CV≤15% indicates good precision of the method.

The results are shown in Tables 3 and 4; the coefficients of variation are both less than 15%, indicating good accuracy.

TABLE 3
Precision results of the gliadin

| Addition amount | Intra-batch variation | Inter-batch variation |
|---|---|---|
| (μg/mL) | CV ± SD(%) | CV ± SD(%) |
| 5 | 0.95 ± 0.21 | 0.66 ± 0.14 |
| 10 | 1.25 ± 0.27 | 0.91 ± 0.20 |
| 20 | 1.54 ± 0.33 | 2.52 ± 0.54 |

TABLE 4
Precision results of the peanut Ara h1 protein

| Addition amount | Intra-batch variation | Inter-batch variation |
|---|---|---|
| (μg/mL) | CV ± SD(%) | CV ± SD(%) |
| 5 | 0.80 ± 0.14 | 0.81 ± 0.13 |
| 10 | 0.44 ± 0.08 | 1.20 ± 0.18 |
| 20 | 0.37 ± 0.06 | 1.45 ± 0.22 |

### Example 4 Detection of β-Lactoglobulin

(1) The β-lactoglobulin was diluted according to a concentration gradient (0.1 μg/mL, 1 g/mL, 10 μg/mL, 25 μg/mL, 50 μg/mL), 100 μL was added to each well of an ELISA plate, and incubated overnight.
(2) 200 μL of PBST (PBS containing 0.1% Tween-20) was added to each well of the ELISA plate, then patted dry. This process was repeated three times to remove unbound target protein.
(3) 200 μL of 5% BSA blocking reagent was added to each well of the ELISA plate, incubating with shaking for 2 h, then the plate was washed three times with PBST washing solution and patted dry.
(4) The aptamer of the above-mentioned β-lactoglobulin was premixed to prepare a premixed solution with a concentration of 20 nM. The sequence of the aptamer of the (3-lactoglobulin is shown in Table 5 below.

TABLE 5
Sequence of the aptamer of the
β-Lactoglobulin

| Allergen | Name | Sequence (5′-3′) |
|---|---|---|
| β- | BLG | CGACGATCGGACCGCA |
| Lactoglobulin | | GTACCCACCCACCAGC |
| | | CCCAACATCATGCCCA |
| | | TCCGTGTGTG |
| | | (SEQ ID NO: 9) |

(5) The premixed solution was added to the ELISA plate after washing away the blocking reagent in step (3). 100 μL of the premixed solution was added to each well, after incubation with shaking for 1 h, the premixed solution was washed away, and the plate was washed 5 times with 200 μL of PBST buffer.
(6) The ultrapure water was added to the ELISA plate, and 50 μL of MilliQ water was added to each well; the ELISA plate was incubated at 95° C. for 12 min to denature the target allergen protein. After the plate cooled to room temperature, 30 μL of supernatant was transferred from each well of the ELISA plate to a centrifuge tube for later use.
(7) The supernatant containing the aptamer was used as a DNA template, and the qPCR amplification and the fluorescence signal detection are performed. In the qPCR detection, the sequences of the probe and primers used for the β-lactoglobulin are shown in Table 6.

TABLE 6
Sequences of probe and primers
of the β-Lactoglobulin

| | | | Amplified |
|---|---|---|---|
| | Primer/ | | Fragment |
| Aptamer | Probe | | Size |
| Name | Name | Sequence (5′-3′) | (bp) |
| β-LG | BLG-F | ATAGGAGTCACGACG | 80 |
| | | ACCAG | |
| | | (SEQ ID NO: 10) | |
| | BLG-R | TCAAGAGGTAGACGC | |
| | | ACATA | |
| | | (SEQ ID NO: 11) | |
| | | ATAGGAGTCACGACG | |
| | BLG-P | ACCAGAGACTAAAAC | |
| | | TCGCCCAGACCGTCA | |
| | | TATTGTAAGTTCAGT | |
| | | TATGTGCGTCTACCT | |
| | | CTTGA | |
| | | (SEQ ID NO: 12) | |

The reaction system of the Taqman qPCR amplification was as follows: total reaction volume of 20 μL, Tagman Probe Master Mix of 10 μL, 10 μM upstream primer of 0.4 μL, 10 M downstream primer of 0.4 μL, 10 μM Tagman Probe of 0.2 μL, DNA template of 1 μL, and sterile water of 8 μL.

The reaction program of the Taqman qPCR amplification was as follows: denaturation at 95° C. for 1 min; denaturation at 95° C. for 10 s, annealing and extension at 60° C. for 30 s, while simultaneously collecting fluorescence signals, for a total of 40 cycles.
(7) The reaction ct value was read and compared with that of the uncoated protein group.

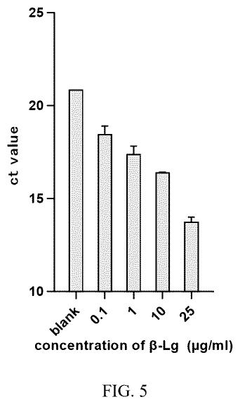

The results are shown in FIG. 5. The ct value of the protein-coated group was significantly lower than that of the uncoated protein group.

In summary, according to embodiments of the present invention, aptamers can specifically recognize target allergen proteins. When the allergen protein denatures and alters its tertiary structure, the aptamer cannot bind to it. By measuring the aptamer content in the system, the content of the target allergen protein is indirectly reflected. This embodiment designed specific probes for gliadin aptamer (G33) and peanut Ara h1 protein aptamer (A1) and determined the experimental conditions, thereby establishing a method for simultaneous detection of two allergens (gliadin and peanut Ara h1 protein) in food. By replacing the target genomic DNA in traditional qPCR methods for detecting allergen proteins with aptamer molecules, the complex and time-consuming target DNA extraction process is eliminated. The method has good sensitivity, enabling simultaneous detection of the content of multiple allergen proteins in food with high efficiency and strong specificity, and can play an important role in food allergen detection and related fields.

In the description of the present specification, references to the terms “one embodiment,” “some embodiments,” “example,” “specific example,” or “some examples,” etc., mean that the specific feature, structure, material, or characteristic described in connection with that embodiment or example is included in at least one embodiment or example of the present invention. In the present specification, the illustrative expressions of the terms used above should not be construed as necessarily referring to the same embodiments or examples. Furthermore, the specific features, structures, materials, or characteristics described may be combined in a suitable manner in any one or more embodiments or examples. Moreover, those skilled in the art can integrate and combine the different embodiments or examples described in the present specification.

Although embodiments of the present invention have been shown and described above, it is understood that the above embodiments are exemplary and should not be construed as limiting the present invention. Those skilled in the art can make changes, modifications, substitutions, and variations to the above embodiments within the scope of the present invention.

What is claimed is:

1. A method for simultaneous detection of multiple allergens in food, comprising the following steps:
(1) diluting a plurality of target allergen proteins in a concentration gradient to obtain diluted allergen proteins, adding the diluted allergen proteins to an enzyme-linked immunosorbent assay (ELISA) plate, and incubating overnight;
(2) washing the ELISA plate with a phosphate-buffered saline containing 0.1% Tween-20 (PBST) washing solution to remove unbound proteins;
(3) adding 5% bovine serum albumin (BSA) blocking reagent to the ELISA plate, incubating with shaking, and then washing away the 5% BSA blocking reagent;
(4) premixing aptamers of the plurality of target allergen proteins into a premixed solution;
(5) adding the premixed solution to the ELISA plate, and incubating with shaking, and then washing away the premixed solution, wherein the 5% BSA blocking reagent is washed away from the ELISA plate in the step (3);
(6) adding ultrapure water to the ELISA plate, incubating at 95° C. to denature the diluted allergen proteins, and recovering a supernatant containing the aptamers;
(7) using the supernatant containing the aptamers as a DNA template, and performing Taqman quantitative polymerase chain reactions (qPCR) amplification and fluorescence signal detection to obtain a reaction ct value; and
(8) plotting a standard curve with the reaction ct value as an ordinate and a target allergen protein concentration as an abscissa.

2. The method for the simultaneous detection of the multiple allergens in the food according to claim 1, wherein the plurality of target allergen proteins comprise gliadin and peanut Ara h1 protein; the nucleotide sequence of an aptamer of the gliadin is shown in SEQ ID NO: 1; and the nucleotide sequence of an aptamer of the peanut Ara h1 protein is shown in SEQ ID NO: 2.

3. The method for the simultaneous detection of the multiple allergens in the food according to claim 2, wherein in the Taqman qPCR amplification and the fluorescence signal detection, a 5′ end of a Tagman probe used for the gliadin is modified with 6-carboxyfluorescein (FAM), and a 3′ end of the Taqman probe used for the gliadin is labeled with a black hole quencher 1 (BHQ1) group; and a 5′ end of a Taqman probe used for the peanut Ara h1 protein is modified with VIC, and a 3′ end of the Taqman probe used for the peanut Ara h1 protein is labeled with the BHQ1 group.

4. The method for the simultaneous detection of the multiple allergens in the food according to claim 3, wherein in the Taqman qPCR amplification and the fluorescence signal detection, the sequences of primers and the sequence of a probe used for the gliadin are shown in SEQ ID NOS: 3-5; and the sequences of primers and the sequence of a probe used for the peanut Ara h1 protein are shown in SEQ ID NOS: 6-8.

5. The method for the simultaneous detection of the multiple allergens in the food according to claim 1, wherein in the step (8), the standard curve satisfies a condition that a correlation coefficient R²≥0.98.

6. The method for the simultaneous detection of the multiple allergens in the food according to claim 1, wherein a reaction system of the Taqman qPCR amplification is as follows: total reaction volume of 20 μL, Tagman Probe Master Mix of 10 μL, 10 μM upstream primer of 0.4 μL, M downstream primer of 0.4 μL, 10 μM Tagman Probe of 0.2 μL, the DNA template of 1 μL, and sterile water of 8 μL; and
a reaction program of the Taqman qPCR amplification is as follows: denaturation at 95° C. for 1 min; the denaturation at 95° C. for 10 s, annealing and extension at 60° C. for 30 s, while simultaneously collecting fluorescence signals, for a total of 40 cycles.

7. The method for the simultaneous detection of the multiple allergens in the food according to claim 1, wherein in the step (3), the 5% BSA blocking reagent is added for incubation with shaking for 2 h.

8. The method for the simultaneous detection of the multiple allergens in the food according to claim 1, wherein in the step (5), the premixed solution is added for incubation with shaking for 1 h.

9. The method for the simultaneous detection of the multiple allergens in the food according to claim 1, wherein in the step (6), the incubating at 95° C. is performed for 12 min.

10. A method for detecting allergens in food, comprising diluting a concentration of a protein to be tested, and obtaining the reaction ct value using the method for the simultaneous detection of the multiple allergens in the food according to claim 1.

11. The method for the simultaneous detection of the multiple allergens in the food according to claim 2, wherein a reaction system of the Taqman qPCR amplification is as follows: total reaction volume of 20 μL, Tagman Probe Master Mix of 10 μL, 10 μM upstream primer of 0.4 μL, M downstream primer of 0.4 μL, 10 μM Tagman Probe of 0.2 μL, the DNA template of 1 μL, and sterile water of 8 μL; and
a reaction program of the Taqman qPCR amplification is as follows: denaturation at 95° C. for 1 min; the denaturation at 95° C. for 10 s, annealing and extension at 60° C. for 30 s, while simultaneously collecting fluorescence signals, for a total of 40 cycles.

12. The method for the simultaneous detection of the multiple allergens in the food according to claim 3, wherein a reaction system of the Taqman qPCR amplification is as follows: total reaction volume of 20 μL, Tagman Probe Master Mix of 10 μL, 10 μM upstream primer of 0.4 μL, M downstream primer of 0.4 μL, 10 μM Tagman Probe of 0.2 μL, the DNA template of 1 μL, and sterile water of 8 μL; and
a reaction program of the Taqman qPCR amplification is as follows: denaturation at 95° C. for 1 min; the denaturation at 95° C. for 10 s, annealing and extension at 60° C. for 30 s, while simultaneously collecting fluorescence signals, for a total of 40 cycles.

13. The method for the simultaneous detection of the multiple allergens in the food according to claim 4, wherein a reaction system of the Taqman qPCR amplification is as follows: total reaction volume of 20 μL, Tagman Probe Master Mix of 10 μL, 10 μM upstream primer of 0.4 μL, M downstream primer of 0.4 μL, 10 μM Tagman Probe of 0.2 μL, the DNA template of 1 μL, and sterile water of 8 μL; and
a reaction program of the Taqman qPCR amplification is as follows: denaturation at 95° C. for 1 min; the denaturation at 95° C. for 10 s, annealing and extension at 60° C. for 30 s, while simultaneously collecting fluorescence signals, for a total of 40 cycles.

14. The method for the simultaneous detection of the multiple allergens in the food according to claim 5, wherein a reaction system of the Taqman qPCR amplification is as follows: total reaction volume of 20 μL, Tagman Probe Master Mix of 10 μL, 10 μM upstream primer of 0.4 μL, M downstream primer of 0.4 μL, 10 μM Tagman Probe of 0.2 μL, the DNA template of 1 μL, and sterile water of 8 μL; and
a reaction program of the Taqman qPCR amplification is as follows: denaturation at 95° C. for 1 min; the denaturation at 95° C. for 10 s, annealing and extension at 60° C. for 30 s, while simultaneously collecting fluorescence signals, for a total of 40 cycles.

15. The method for detecting the allergens in the food according to claim 10, wherein in the method for the simultaneous detection of the multiple allergens in the food, the plurality of target allergen proteins comprise gliadin and peanut Ara h1 protein; the nucleotide sequence of an aptamer of the gliadin is shown in SEQ ID NO: 1; and the nucleotide sequence of an aptamer of the peanut Ara h1 protein is shown in SEQ ID NO: 2.

16. The method for detecting the allergens in the food according to claim 15, wherein in the Taqman qPCR amplification and the fluorescence signal detection of the method for the simultaneous detection of the multiple allergens in the food, a 5′ end of a Taqman probe used for the gliadin is modified with FAM, and a 3′ end of the Taqman probe used for the gliadin is labeled with a BHQ1 group; and a 5′ end of a Taqman probe used for the peanut Ara h1 protein is modified with VIC, and a 3′ end of the Taqman probe used for the peanut Ara h1 protein is labeled with the BHQ1 group.

17. The method for detecting the allergens in the food according to claim 16, wherein in the Taqman qPCR amplification and the fluorescence signal detection of the method for the simultaneous detection of the multiple allergens in the food, the sequences of primers and the sequence of a probe used for the gliadin are shown in SEQ ID NOS: 3-5; and the sequences of primers and the sequence of a probe used for the peanut Ara h1 protein are shown in SEQ ID NOS: 6-8.

18. The method for detecting the allergens in the food according to claim 10, wherein in the step (8) of the method for the simultaneous detection of the multiple allergens in the food, the standard curve satisfies a condition that a correlation coefficient R²≥0.98.

19. The method for detecting the allergens in the food according to claim 10, wherein in the method for the simultaneous detection of the multiple allergens in the food, a reaction system of the Taqman qPCR amplification is as follows: total reaction volume of 20 μL, Tagman Probe Master Mix of 10 μL, 10 μM upstream primer of 0.4 μL, 10 μM downstream primer of 0.4 μL, 10 M Tagman Probe of 0.2 μL, the DNA template of 1 μL, and sterile water of 8 μL; and
a reaction program of the Taqman qPCR amplification is as follows: denaturation at 95° C. for 1 min; the denaturation at 95° C. for 10 s, annealing and extension at 60° C. for 30 s, while simultaneously collecting fluorescence signals, for a total of 40 cycles.

20. The method for detecting the allergens in the food according to claim 10, wherein in the step (3) of the method for the simultaneous detection of the multiple allergens in the food, the 5% BSA blocking reagent is added for incubation with shaking for 2 h.
